(19) United States
(12) Patent Application Publication
(10) Pub. No.: US 2015/0164319 A1
(43) Pub. Date: Jun. 18, 2015

(54) PUPIL DETECTING APPARATUS AND PUPIL DETECTING METHOD

(71) Applicant: HYUNDAI MOTOR COMPANY, Seoul (KR)

(72) Inventors: Yang Shin KIM, Tongyeong-si (KR); Seok Beom LEE, Seoul (KR); Dong Hee SEOK, Seoul (KR)

(21) Appl. No.: 14/520,243

(22) Filed: Oct. 21, 2014

(30) Foreign Application Priority Data
Dec. 17, 2013 (KR) 10-2013-0157426

(51) Int. Cl. A61B 3/113

(52) U.S. Cl. CPC A61B 3/113

(57) ABSTRACT

A pupil detecting apparatus includes a first detector configured to binarize an image including an eye region according to brightness values and detect a pupil region from the image including an eye region, a second detector configured to detect edges from the image including an eye region and detect a pupil boundary region by using the detected edges, and a pupil detector configured to determine a final pupil region by using the detected pupil region and the detected pupil boundary region.

### CROSS-REFERENCE TO RELATED APPLICATION

This application is based on and claims priority to Korean Patent Application No. 10-2013-0157426, filed on Dec. 17, 2013 in the Korean Intellectual Property Office, the entire content of which is incorporated herein by reference.

### TECHNICAL FIELD

The present inventive concept relates to a pupil detecting apparatus and a pupil detecting method for detecting a pupil from an image including an eye region.

### BACKGROUND

As the electronic technologies have advanced, various electronic devices have been used in daily lives and research into human computer interaction (HCl) has been actively conducted.

In particular, HCl using a movement of a user pupil advantageously provides convenience and supports a high input speed. Thus, techniques of tracking a user's gaze to analyze a psychological state have also been developed.

In order to track a user's gaze, a process of detecting a face region from an image obtained by imaging a user, detecting an eye region, and subsequently detecting a pupil from the detected eye region needs to be performed. The eye region is a very small area in the entire image, so resolution of the eye region is low. Thus, if resolution of an initial image is not high, it may be difficult to accurately detect a pupil region and the center of a pupil.

### SUMMARY

Accordingly, the present inventive concept has been made to solve the above-mentioned problems occurring in the prior art while advantages achieved by the prior art are maintained intact.

An aspect of the present disclosure provides a pupil detecting apparatus and a pupil detecting method capable of accurately detecting a pupil even from an image with low resolution.

One aspect of the present disclosure relates to a pupil detecting apparatus including a first detector, a second detector and a pupil detector. The first detector is configured to binarize an image including an eye region according to brightness values and detect a pupil region from the image including an eye region. The second detector is configured to detect edges from the image including an eye region and detect a pupil boundary region by using the detected edges. The pupil detector is configured to determine a final pupil region by using the detected pupil region and the detected pupil boundary region.

The pupil detector may include a pupil candidate point detector configured to detect pupil candidate points by using the detected pupil region and the pupil boundary region, and a pupil region determiner configured to determine the final pupil region by applying ellipse fitting using random sample consensus (RANSAC) to the pupil candidate points.

The pupil candidate point detector may be configured to detect, as the pupil candidate points, a region of the pupil boundary region which overlaps with the pupil region.

Another aspect of the present inventive concept encompasses a pupil detecting method including binarizing an image including an eye region according to brightness values and detecting a pupil region from the image including an eye region. According to the pupil detecting method, edges are detected from the image including an eye region and detecting a pupil boundary region by using the detected edges. A final pupil region is determined by using the detected pupil region and the detected pupil boundary region.

In the determining of the final pupil region, pupil candidate points may be determined by using the detected pupil region and the detected pupil boundary region, and the final pupil region may be determined by applying ellipse fitting using random sample consensus (RANSAC) to the pupil candidate points.

In the detecting of the pupil candidate points, a region of the pupil boundary region which overlaps with the pupil region may be detected as the pupil candidate points.

### BRIEF DESCRIPTION OF THE DRAWINGS

The above and other objects, features and advantages of the present inventive concept will be more apparent from the following detailed description taken in conjunction with the accompanying drawings, in which like reference characters may refer to the same or similar parts throughout the different views. The drawings are not necessarily to scale, emphasis instead being placed upon illustrating the principles of the embodiments of the inventive concept.

FIG. 1 is a block diagram illustrating a configuration of a pupil detecting apparatus according to an exemplary embodiment of the present inventive concept.

FIG. 2 is a block diagram illustrating a detailed configuration of a pupil detector according to an exemplary embodiment of the present inventive concept.

FIGS. 3A and 3B each show a view illustrating a detected pupil region according to an exemplary embodiment of the present inventive concept.

FIG. 4 is a view illustrating a central point of a detected pupil according to an exemplary embodiment of the present inventive concept.

FIG. 5 is a flow chart illustrating a pupil detecting method according to an exemplary embodiment of the present inventive concept.

### DETAILED DESCRIPTION

Hereinafter, exemplary embodiments of the present inventive concept will be described in detail with reference to the accompanying drawings.

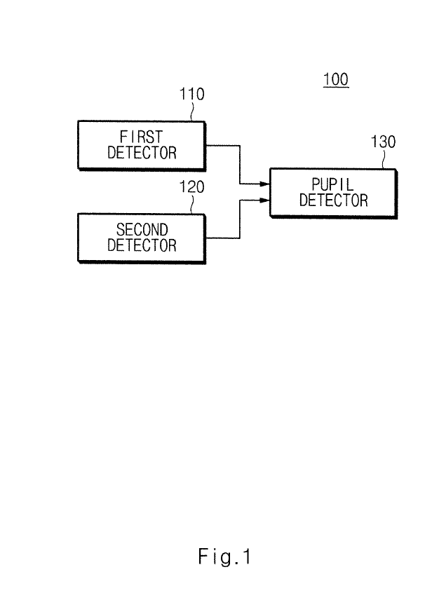

FIG. 1 is a block diagram illustrating a configuration of a pupil detecting apparatus according to an exemplary embodiment of the present inventive concept.

Referring to FIG. 1, a pupil detecting apparatus 100 may include a first detector 110, a second detector 120, and a pupil detector 130.

The first detector 110 may detect a pupil from an image including an eye region. In detail, the first detector 110 may binarize an image including an eye region according to brightness values and detect a pupil region therefrom.

The first detector 110 may binarize the image including an eye region according to a pre-set threshold value. Namely, the first detector 110 may divide pixels included in the image into a group having a brightness value greater than or equal to the threshold value and a group having a brightness value smaller than the threshold value. The pupil may be a region appearing to be the darkest around an eye, so a pupil region may be detected by setting a threshold value in consideration of such features.

However, in the case of binarizing the image according to brightness values and detecting a pupil region, a pupil region having a size different from that of an actual or normal pupil region may be detected according to a threshold value. For example, if a threshold value is set to be smaller than a normal threshold value to detect a actual or normal pupil region, a pupil region smaller than the actual or normal pupil region may be detected, and if a threshold value is set to be greater than the normal threshold value, a pupil region greater than the actual or normal pupil region may be detected.

Also, when an eyeline is drawn in an eye such that an eye boundary has a brightness value similar to that of a pupil region or when an image is captured in a dark environment to have a low brightness value overall, a region other than the pupil may be detected as a pupil region.

The second detector 120 may detect a pupil from the image including an eye region. In detail, the second detector 120 may detect an edge from the image including an eye region and detect a pupil boundary region by using the detected edge.

Since a pupil appears to be dark, relative to an iris, the second detector 120 may detect a boundary region of the pupil by using a difference in brightness. The second detector 120 may detect edges in which a brightness value is changed by more than a pre-set value in the image including an eye region. The second detector 120 may detect a circular or oval edge, among the detected edges, as a boundary region of the pupil.

Meanwhile, the image including an eye region inputted to the first detector 110 and the second detector 120 may be captured by an infrared camera.

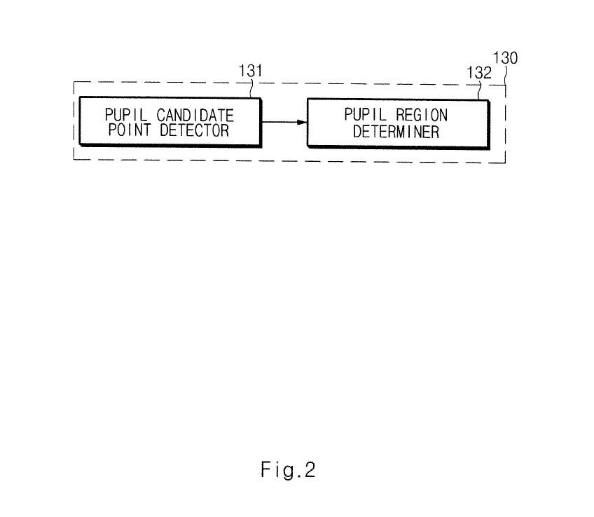

The pupil detector 130 may determine a final pupil region by using the pupil region and the pupil boundary region, detected by the first detector 110 and the second detector 120. The pupil detector 130 will be described in detail with reference to FIG. 2.

FIG. 2 is a block diagram illustrating a detailed configuration of a pupil detector according to an exemplary embodiment of the present inventive concept.

Referring to FIG. 2, the pupil detector 130 may include a pupil candidate point detector 131 and a pupil region determiner 132.

The pupil candidate point detector 131 may detect pupil candidate points by using the pupil region and the pupil boundary region, detected by the first detector 110 and the second detector 120. The pupil candidate point detector 131 may detect, as pupil candidate points, a region of the pupil boundary region detected by the second detector 120, which overlaps with the pupil region detected by the first detector 110. Namely, the pupil candidate point detector 131 may detect, as pupil candidate points, pixels which are detected simultaneously or commonly by the first detector 110 and the second detector 120.

The pupil region determiner 132 may determine a final pupil region by using the pupil candidate points detected by the pupil candidate point detector 131. The pupil region determiner 132 may determine a pupil region by applying ellipse fitting using random sample consensus (RANSAC) to the pupil candidate points detected by the pupil candidate point detector 131.

The pupil candidate points detected by the pupil candidate point detector 131 may not be detected as having a circular or oval shape as an accurately closed curve. Namely, a partial region of the detected pupil candidate points may be discontinuous, or a region other than a pupil may be detected. In order to detect a pupil region having a circular or oval shape as a closed curve, excluding erroneously detected pupil candidate points, the pupil region determiner 132 may use the RANSAC or other ellipse fitting algorithm.

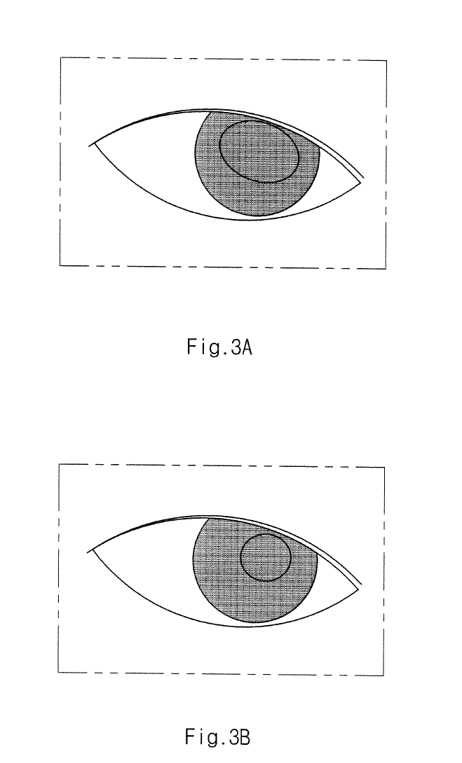

FIGS. 3A and 3B each show a view illustrating a detected pupil region according to an exemplary embodiment of the present inventive concept.

FIG. 3A illustrates a pupil region detected by simply binarizing the image according to brightness values, and FIG. 3B illustrates a pupil region detected according to an embodiment of the present inventive concept. It can be seen that, in the case of FIG. 3A, the detected pupil region is greater than the actual pupil region, causing a great error, but in the case of FIG. 3B, the detected pupil region is similar to the actual pupil region.

Namely, according to an embodiment of the present inventive concept, even when a boundary between an iris and a pupil is not clear due to a change in ambient illumination or low resolution, the pupil region may be accurately detected.

Meanwhile, the pupil detecting apparatus 100 may further include a pupil central point detector (not shown). The pupil central point detector (not shown) may detect a pupil central point by using the pupil region detected by the pupil detector 130. The pupil central point detector (not shown) may detect a middle point or the center of gravity of the pupil region detected by the pupil detector 130, as a pupil central point.

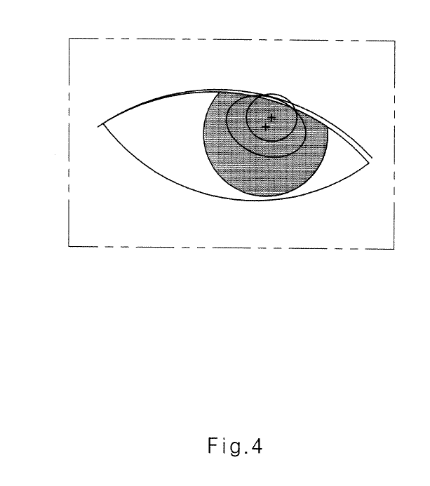

FIG. 4 is a view illustrating a central point of a detected pupil according to an exemplary embodiment of the present inventive concept.

Referring to FIG. 4, pupil central points detected based on the detected pupil regions of FIGS. 3A and 3B. When the two pupil central points illustrated in FIG. 4 are compared, in the case in which the pupil central point is detected by using the pupil region detected by binarizing the image according to brightness values, a point different from the actual pupil center may be detected as a pupil central point. In contrast, in an embodiment of the present inventive concept, a point similar to the actual pupil center may be detected as a pupil central point.

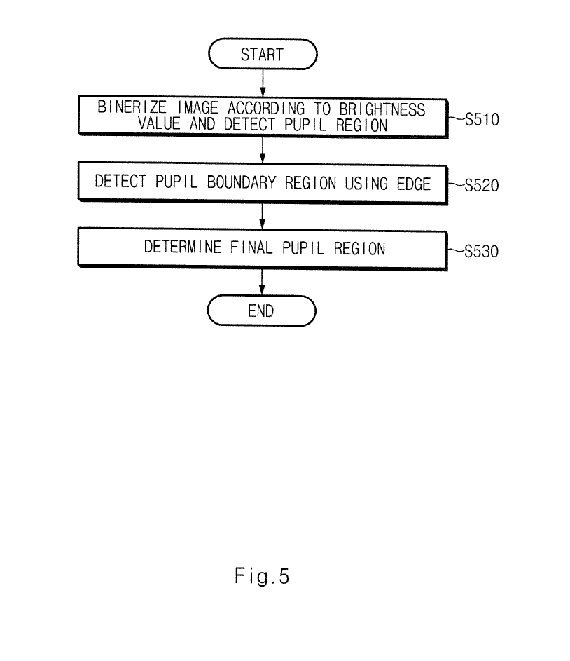

FIG. 5 is a flow chart illustrating a pupil detecting method according to an exemplary embodiment of the present inventive concept.

Referring to FIG. 5, the pupil detecting apparatus 100 may detect a pupil region by binarizing an image including an eye region according to brightness values (S510). Pixels included in the image may be divided into a group having a brightness value greater than or equal to a threshold value and a group having a brightness value smaller than the threshold value. Since the pupil is a region appearing to be the darkest around an eye, a pupil region may be detected by setting a threshold value in consideration of such features.

Edges may be detected from the image including an eye region, and a pupil boundary region may be detected by using the detected edges (S520). Since a pupil appears to be dark, relative to an iris, a boundary region of the pupil may be detected by using a difference in brightness between the pupil and the iris.

A final pupil region may be determined by using the detected pupil region and the detected pupil boundary region (S530). Pupil candidate points may be detected by using the detected pupil region and the detected pupil boundary region, and a pupil region may be determined by applying ellipse fitting using RANSAC to the detected pupil candidate points.

In detecting the pupil candidate points, a region of the detected pupil boundary region, which overlaps with the pupil region detected in operation S510 may be detected as pupil candidate points.

Meanwhile, in FIG. 5, the pupil region may be detected and the pupil boundary region may be subsequently detected, but operations S510 and S520 may be performed simultaneously or irrespective of order.

When the final pupil region is detected, a pupil central point may be detected by using the detected pupil region. In detail, a middle point or the center of gravity of the detected pupil region may be detected as a pupil central point.

The pupil detecting method according to various exemplary embodiments of the present inventive concept as described above may be implemented as an electronic device-executable program that can be executed in an electronic device, e.g., a microprocessor or a dedicated hardware device. Such a program may be stored in various recording mediums and used.

In detail, codes for performing the foregoing methods may be stored in various types of non-volatile recording mediums such as a flash memory, a read-only memory (ROM), an erasable programmable ROM (EPROM), an electrically erasable and programmable ROM (EEPROM), a hard disk, a removable disk, a memory card, a USB memory, a CD-ROM, or the like.

According to an exemplary embodiment of the present inventive concept, even when a boundary between an iris and a pupil is not clear due to a change in ambient illumination or low resolution, a pupil region may be accurately detected from an input eye image.

It should be interpreted that the scope of the present inventive concept is defined by the following claims rather than the above-mentioned detailed description and all modifications or alterations deduced from the meaning, the scope, and equivalences of the claims are included in the scope of the present inventive concept.

What is claimed is:

1. A pupil detecting apparatus, comprising:
a first detector configured to binarize an image including an eye region according to brightness values and detect a pupil region from the image including an eye region;
a second detector configured to detect edges from the image including an eye region and detect a pupil boundary region by using the detected edges; and
a pupil detector configured to determine a final pupil region by using the detected pupil region and the detected pupil boundary region.

2. The pupil detecting apparatus according to claim 1, wherein the pupil detector includes:
a pupil candidate point detector configured to detect pupil candidate points by using the detected pupil region and the detected pupil boundary region; and
a pupil region determiner configured to determine the final pupil region by applying ellipse fitting using random sample consensus (RANSAC) to the pupil candidate points.

3. The pupil detecting apparatus according to claim 2, wherein the pupil candidate point detector is configured to detect, as the pupil candidate points, a region of the pupil boundary region which overlaps with the pupil region.

4. A pupil detecting method, comprising:
binarizing an image including an eye region according to brightness values and detecting a pupil region from the image including an eye region;
detecting edges from the image including an eye and detecting a pupil boundary region by using the detected edges; and
determining a final pupil region by using the detected pupil region and the detected pupil boundary region.

5. The pupil detecting method according to claim 4, wherein the determining of the final pupil region includes:
detecting pupil candidate points by using the detected pupil region and the detected pupil boundary region; and
determining the final pupil region by applying ellipse fitting using random sample consensus (RANSAC) to the pupil candidate points.

6. The pupil detecting method according to claim 5, wherein the detecting of the pupil candidate points includes detecting, as the pupil candidate points, a region of the pupil boundary region which overlaps with the pupil region.

7. A non-transitory computer-readable recording medium comprising computer executable instructions configured to perform the method according to claim 4.
